(19) United States
(12) Patent Application Publication
(10) Pub. No.: US 2004/0087612 A1
(43) Pub. Date: May 6, 2004

(54) Method of increasing lacrimal gland tearing by topical administration of tacrolimus

(76) Inventors: Robert V. English, Cary, NC (US); Bradley Nadelstein, Chesapeake, VA (US)

Correspondence Address: Peter J. Van Bergen, 402 West Duke of Gloucester St., Williamsburg, VA, 23185, US

(21) Appl. No.: 10/287,052

(22) Filed: Nov. 4, 2002

(51) Int. Cl. A61K 31/4745; A61K 31/7012; A61K 31/716

(52) U.S. Cl. 514/291; 514/57; 514/53

(57) ABSTRACT

A method is provided for increasing lacrimal gland tearing in a patient's eye. Specifically, tacrolimus in a pharmaceutically acceptable vehicle is topically administered to the patient's eye.

### FIELD OF THE INVENTION

The invention relates generally to methods of improving lacrimal gland tear production, and more particularly to a method of increasing lacrimal gland tearing by the topical administration of tacrolimus to the patient's eye.

### BACKGROUND OF THE INVENTION

In both human and animal eyes, the cornea is optically clear living tissue that allows light to enter the eye. As living tissue, the cornea requires a supply of oxygen and nutrient's which, for most living tissue, is delivered by blood vessels running therethrough. However, a healthy cornea (i.e. an optically, clear cornea) has no blood vessels. Instead, the cornea is partly supplied with oxygen and nutrients through a three-layer tear film on the cornea's surface. The tear film's outer layer is an oily layer supplied by glands in the eyelids. The middle layer is an aqueous layer produced by the lacrimal gland associated with the eye. The innermost layer is a mucous layer produced by glands that are in the folds of the eyelid.

The eye's tear film is critical in maintaining the health of the eye's cornea and, therefore, its optical clarity. However, malfunctions in the tear film can cause problems ranging from discomfort to loss of vision. One of the most notable malfunctions occurs in the eye's lacrimal gland that is responsible for producing tears that form the tear film's middle aqueous layer. Reduced lacrimal gland output leading to a tear deficiency in the tear film's middle layer causes a condition known as “dry eye” (i.e., keratoconjunctivitis sicca). At a minimum, this condition causes dryness, discomfort and blurred vision. However, in more advanced or severe cases, reduced lacrimal gland output causes a breakdown in the tear film's ability to deliver oxygen and nutrients to the cornea. This results in destructive changes to include brown pigmentation of the cornea, fibrosis or scar tissue formation, ulcer development on the cornea, and blood vessel growth across the cornea. Thus, overall eye comfort and visual acuity are linked to lacrimal gland tearing.

As disclosed in U.S. Pat. No. 4,839,342, topical use of the T-cell immunosuppressive compound cyclosporin has been shown to increase tear production in dogs. While the method of action is unclear, patients that have been topically treated with cyclosporin have experienced an increase in tear production. However, the use of topical cyclosporin is not effective in all cases and can cause topical irritation preventing use of the compound. Further, the cyclosporin is mixed in oil-based suspensions that can cause irritation and blurred vision.

### SUMMARY OF THE INVENTION

Accordingly, it is an object of the present invention to provide a method for increasing lacrimal gland tearing.

Other objects and advantages of the present invention will become more obvious hereinafter in the specification and drawings.

In accordance with the present invention, a method for increasing lacrimal gland tearing comprises the step of topically administering tacrolimus to a patient's eye in a pharmaceutically acceptable vehicle.

### DETAILED DESCRIPTION OF THE INVENTION

The present invention will be understood more clearly and fully from the following description of preferred embodiments thereof.

The present invention provides a method for increasing lacrimal gland tearing in order to increase tear production in a patient's eye. The method is especially useful for increasing tear production when a patient's eye is tear deficient to the point that the patient suffers from keratoconjunctivitis sicca (or KCS as it will be referred to hereinafter). The method includes the step of topically administering tacrolimus to the patient's eye. It has been found that such topical administration of tacrolimus increases lacrimal gland tearing and is, therefore, useful for treating tear deficiencies, as is the case for a patient suffering with KCS.

Prior to the method disclosed herein, there has been no teaching or suggestion to use tacrolimus topically or systemically to treat a patient's lacrimal gland dysfunction that results in a tear deficiency such as that found in a patient suffering with KCS.

Tacrolimus (i.e., compound number FK 506) is a macrolide compound produced by, for example, microorganisms of the genus Streptyomyces, such as Streptomyces tsukabaensis No. 9993 [deposited with National Institute of Bioscience and Human Technology Agency of Industrial Science and Technology (formerly Fermentation Research Institute Agency of Industrial Science and Technology) at 1-3, Higashi 1-chome, Tsukuba-shi, Ibaraki, Japan date of deposit Oct. 5, 1984, accession number FERM BP-927]. However, as would be well understood by one of ordinary skill in the art, the particular formulation of tacrolimus can also be produced using other species of Streptomyces, the choice of which is not a limitation of the present invention.

Tacrolimus is a potent inhibitor of T-cell immune functions and is currently used for the treatment of immune-mediated disorders in humans and animals. Such disorders can occur as a result of organ or tissue transplants, grafts, autoimmune diseases (e.g., lupus, rheumatoid arthritis, diabetes mellitus, myasthenia gravis, multiple sclerosis, etc.), inflammation (e.g., caused by conditions such as psoriasis, dermatitis, eczema, seborrhea, bowel disease, emphysema, acute respiratory distress syndrome, bronchitis and the like), and uveitis. In addition, tacrolimus has been considered in the treatment of solid tumors, adult T-cell leukemia/lymphoma, fungal infections, and hyper-proliferative vascular diseases such as rectenosis and atherosclerosis. However, preparation and use of tacrolimus as a typical ophthalmic medication for increasing lacrimal gland tearing has not been previously described or suggested.

In accordance with the present invention, tacrolimus can be used in any efficacious concentration in a pharmaceutically acceptable vehicle. The tacrolimus is advantageously administered topically as an ophthalmic (solution or suspension) drop. Tacrolimus concentrations in the solution or suspension range from 0.01-0.04 percent weight/volume.

In one embodiment of the present invention, the efficacious concentration of tacrolimus is mixed in an aqueous solution or suspension. In general, the aqueous solution or suspension includes tacrolimus and water, and can include one or more pharmaceutically acceptable excipients added to give the ultimate medication preparation its desired consistency or form. For topical administration of the tacrolimus-based medication on a patient's eye, it is preferred that the aqueous solution be one that stays in place on the patient's eye to allow the effects of tacrolimus to be fully realized. Thus, in the present invention, the pharmaceutically acceptable excipient(s) can be one or more inert thickening agents. Other inert excipients such as colors, stabilizers, preservatives, etc., can be included as needed without departing from the scope of the present invention.

In the present invention, tacrolimus is typically administered topically as an aqueous ophthalmic suspension that can be in the form of drops or an ointment. While the particular mixture and efficacious concentration of tacrolimus can be varied, one such suspension used in the treatment of a number of dogs included the following:

i) Approximately 0.02 percent weight/volume tacrolimus crystals,

ii) Approximately 0.2 percent weight/volume hydroxypropyl methylcellulose,

iii) Approximately 0.4 percent weight/volume lactose, and

iv) Sterile, deionized water mixed with the first three ingredients in an amount sufficient to achieve the above-noted concentrations.

Hydroxypropyl methylcellulose and lactose are common inert thickening agents used in ophthalmic preparations. The use of deionized water insures that no dissociation of the tacrolimus molecules will occur during solution/suspension preparation.

Preparation of the suspension involves mixing the above four ingredients while heating same to approximately 120° F. If prepared at the pharmacy level, the mixture can be repeatedly passed through a small-holed (e.g., on the order of a 25 gauge) dropper or needle to assure uniform suspension of the mixture's solid particles.

The following examples illustrate the effectiveness of the treatment method of the present invention. Specifically, eleven different dogs of varying breeds and ages were treated using the above solution for treatment periods ranging from 20-84 days. The eleven dogs had naturally occurring keratoconjunctivitis sicca. The subject dogs received one drop of the above-described tacrolimus-based suspension topically in the eye, twice a day. Prior to therapy, tear production was measured using standard Schirmer tear quantification. Corneal fibrosis, vascularization and pigmentation were measured using slit lamp biomicroscopy and fundus reflection during indirect ophthalmoscopy. Complete physical examinations were preformed by licensed veterinarian ophthalmologists. These procedures were repeated at 2 weeks, 4 weeks and 8 to 12 weeks post initiation of therapy. Owners were interviewed and questioned at each examination period to determine if noxious or irritant reactions to the above-described tacrolimus solution preparation were observed during the course of therapy. No other tear stimulation therapy was administered. The results are summarized in the table below.

| Breed | Age | T/L OD | T/L OS | No. Days | T/L OD | T/L OS |
|---|---|---|---|---|---|---|
| Boxer | 6.8 | 8 | 13 | 29 | 12 | 14 |
| Gldn. | 12.8 | 7 | 5 | 24 | 7 | 11 |
| Rtrvr. | | | | | | |
| Cocker | 11.8 | 7 | 9 | 35 | 13 | 13 |
| Spaniel | | | | | | |
| Shih Tzu | 4.4 | 9 | 12 | 35 | 15 | 19 |
| Shih Tzu | 11.7 | 11 | 13 | 28 | 17 | 15 |
| Shih Tzu | 4.9 | 15 | 15 | 28 | 18 | 18 |
| Lhasa | 6.6 | 6 | 13 | 84 | 14 | 17 |
| Apso | | | | | | |
| Yorkshire | 1.3 | 12 | 6 | 33 | 18 | 14 |
| Terrier | | | | | | |
| Yorkshire | 15.2 | 3 | 7 | 32 | 15 | 15 |
| Terrier | | | | | | |
| Jack Russell | 3.9 | 15 | 7 | 20 | 16 | 16 |
| Terrier | | | | | | |
| Cocker | 12.8 | 12 | 13 | 29 | 16 | 18 |
| Spaniel | | | | | | |

In the table, “T/L OD” refers to tear levels in the right eye, “T/L OS” refers to tear levels in the left eye, and “No. days” is the number of days that treatment was received.

The advantages of the present invention are numerous. Lacrimal gland tearing is increased by topically administering tacrolimus solution to a patient's eye. In the described embodiment, an aqueous solution is used thereby eliminating the problems associated with oil-based topical ointments such as those made with cyclosporin. However, it is to be understood that the present invention can also be practiced using a pharmaceutically acceptable vehicle that is oil-based when an application requires same.

Although the invention has been described relative to a specific embodiment thereof, there are numerous variations and modifications that will be readily apparent to those skilled in the art in light of the above teachings. It is therefore to be understood that, within the scope of the appended claims, the invention may be practiced other than as specifically described.

What is claimed as new and desired to be secured by Letters Patent of the United States is:

1. A method for increasing lacrimal gland tearing comprising the step of topically administering tacrolimus to a patient's eye in a pharmaceutically acceptable vehicle.

2. A method according to claim 1 wherein said tacrolimus is administered in an aqueous solution comprising approximately 0.01-0.04 percent weight/volume of tacrolimus therein.

3. A method according to claim 2 wherein said aqueous solution includes water and at least one pharmaceutically acceptable excipient.

4. A method according to claim 3 wherein said water is deionized water.

5. A method according to claim 3 wherein said at least one pharmaceutically acceptable excipient includes at least one thickening agent.

6. A method according to claim 5 wherein said at least one thickening agent includes hydroxypropyl methylcellulose and lactose.

7. A method according to claim 1 for increasing tear production in a tear-deficient eye of the patient comprising the step of topically administering a therapeutically efficient amount of tacrolimus to said eye.

8. A method according to claim 7 wherein said tacrolimus is administered in an aqueous solution comprising approximately 0.01-0.04 percent weight/volume of tacrolimus therein.

9. A method according to claim 8 wherein said aqueous solution includes water and at least one pharmaceutically acceptable excipient.

10. A method according to claim 8 wherein said water is deionized water.

11. A method according to claim 8 wherein said at least one pharmaceutically acceptable excipient includes at least one thickening agent.

12. A method according to claim 11 wherein said at least one thickening agent includes hydroxypropyl methylcellulose and lactose.

13. A method according to claim 1 for treating keratoconjunctivitis sicca in the patient comprising the step of administering a therapeutically efficient amount of tacrolimus topically to the patient's eye affected with keratoconjunctivitis sicca.

14. A method according to claim 13 wherein said tacrolimus is administered in an aqueous solution comprising approximately 0.01-0.04 percent weight/volume of tacrolimus therein.

15. A method according to claim 14 wherein said aqueous solution includes water and at least one pharmaceutically acceptable excipient.

16. A method according to claim 14 wherein said water is deionized water.

17. A method according to claim 14 wherein said at least one pharmaceutically acceptable excipient includes at least one thickening agent.

18. A method according to claim 17 wherein said at least one thickening agent includes hydroxypropyl methylcellulose and lactose.

19. A method for increasing lacrimal gland tearing comprising the step of topically administering an aqueous ophthalmic solution to a patient's eye, said aqueous ophthalmic solution including approximately 0.02 percent weight/volume of tacrolimus, at least one pharmaceutically acceptable thickening agent, and deionized water.

20. A method according to claim 19 wherein said at least one pharmaceutically acceptable thickening agent includes hydroxypropyl methylcellulose and lactose.

21. A method for increasing tear production in a tear-deficient eye of the patient comprising the step of topically administering an aqueous ophthalmic solution to the patient's tear-deficient eye, said aqueous ophthalmic solution including approximately 0.02 percent weight/volume of tacrolimus, at least one pharmaceutically acceptable thickening agent, and deionized water.

22. A method according to claim 20 wherein said at least one pharmaceutically acceptable thickening agent includes hydroxypropyl methylcellulose and lactose.

23. A method for treating keratoconjunctivitis sicca in a patient comprising the step of topically administering an aqueous ophthalmic solution to the patient's eye affected with keratoconjunctivitis sicca, said aqueous ophthalmic solution including approximately 0.02 percent weight/volume of tacrolimus, at least one pharmaceutically acceptable thickening agent, and deionized water.

24. A method according to claim 23 wherein said at least one pharmaceutically acceptable thickening agent includes hydroxypropyl methylcellulose and lactose.
